(19) United States
(12) Patent Application Publication
(10) Pub. No.: US 2026/0069166 A1
(43) Pub. Date: Mar. 12, 2026

(54) Cardiovascular Biomarker Estimation Using PPG And BCG from a Wearable Device

(71) Applicant: Samsung Electronics Co., Ltd., Suwon-si (KR)

(72) Inventors: Mehrab Bin Morshed, Gilroy, CA (US); Md Mahbubur Rahman, San Jose, CA (US); Li Zhu, Saratoga, CA (US); Hao Zhou, Sunnyvale, CA (US); Larry Zhang, San Jose, CA (US)

(21) Appl. No.: 19/318,933

(22) Filed: Sep. 4, 2025

Related U.S. Application Data
(60) Provisional application No. 63/692,641, filed on Sep. 9, 2024.
(60) Provisional application No. 63/801,645, filed on May 7, 2025.

(51) Int. Cl. A61B 5/11; A61B 5/00; A61B 5/024

(52) U.S. Cl. CPC A61B 5/1102; A61B 5/02416; A61B 5/7246; A61B 5/7264; A61B 2562/0219

(57) ABSTRACT

In one embodiment, a method includes accessing (1) a BCG signal created by a wearable-device IMU sensor and (2) a PPG signal created by a wearable-device PPG sensor; and determining, from the BCG signal and the PPG signal, one or more deep features related to a cardiovascular biomarker. The method further includes comparing (1) at least some of the BCG signal to a BCG signal template and (2) at least a some of the PPG signal to a PPG signal template; determining, based on the comparison, (1) a high-quality BCG signal portion that corresponds to the BCG signal template and (2) a high-quality PPG signal portion that corresponds to the PPG signal template; determining one or more morphological features from at least one of the high-quality BCG signal and the high-quality PPG signal; and determining one or more cardiovascular biomarkers from the deep features and the morphological features.

### PRIORITY CLAIM

This application claims the benefit under 35 U.S.C. § 119 of U.S. Provisional Patent Application Nos. 63/692,641 filed Sep. 9, 2024 and 63/801,645 filed May 7, 2025, each of which is incorporated by reference herein.

### TECHNICAL FIELD

This application generally relates to cardiovascular biomarker estimation using photoplethysmography (PPG) and ballistocardiogram (BCG) from a wearable device.

### BACKGROUND

Cardiovascular diseases remain leading causes of global morbidity and mortality. Cardiac output is the estimate of how much blood is ejected from heart in a minute, and is often used an indicator for understanding symptoms of heart failure, atrial fibrillation, acute mental stress, and blood pressure. Continuous monitoring of cardiac biomarkers such as cardiac output is crucial for early detection, effective management, and prevention of cardiovascular complications, such as stroke, heart failure, and coronary artery disease. In addition, cardiovascular biomarkers such as cardiac output can be used to estimate stress (e.g., eustress vs. distress) and other physiological phenomena.

A variety of wearable devices in the consumer space could be used to track cardiovascular biomarkers, such as cardiac output, but such biomarkers can be difficult to accurately detect with consumer wearable devices.

### BRIEF DESCRIPTION OF THE DRAWINGS

FIG. 1 illustrates an example method for estimating a cardiovascular biomarker from PPG signals and BCG signals obtained from sensors of one or more wearable devices.

FIG. 2 illustrates an example implementation of FIG. 1 that uses a trained temporal convolution network.

FIG. 3 illustrates an example process for estimating cardiac output from PPG data only.

FIG. 4 illustrates examples of PPG waves classified as “excellent” and PPG waves classified as “acceptable.”

FIG. 5 illustrates an example process for estimating CO based only on BCG data.

FIG. 6 illustrates an example computing system.

### DESCRIPTION OF EXAMPLE EMBODIMENTS

Photoplethysmogram (PPG) and ballistocardiogram (BCG) provides important information about cardiac activities of individuals, and these sensors may be integrated into one or more wearable devices. However, accurately estimating biomarkers such as cardiac output from such sensors in wearable devices can be challenging.

The techniques of this disclose provide accurate cardiac biomarker detection based on PPG signals obtained from a wearable device, BCG signals obtained from a wearable device, or a combination thereof. For instance, an IMU and a PPG can be integrated in one or more wearable devices such as earbuds, watches, rings, bracelets, necklaces, glasses, etc. Certain embodiments of this disclosure use both the BCG signal and the PPG signal obtained from a wearable device to estimate cardiac biomarkers. As explained below, other embodiments use only a PPG signal or a BCG signal to estimate such biomarkers (e.g., in order to reduce the power demands required to obtain both PPG and BCG signals).

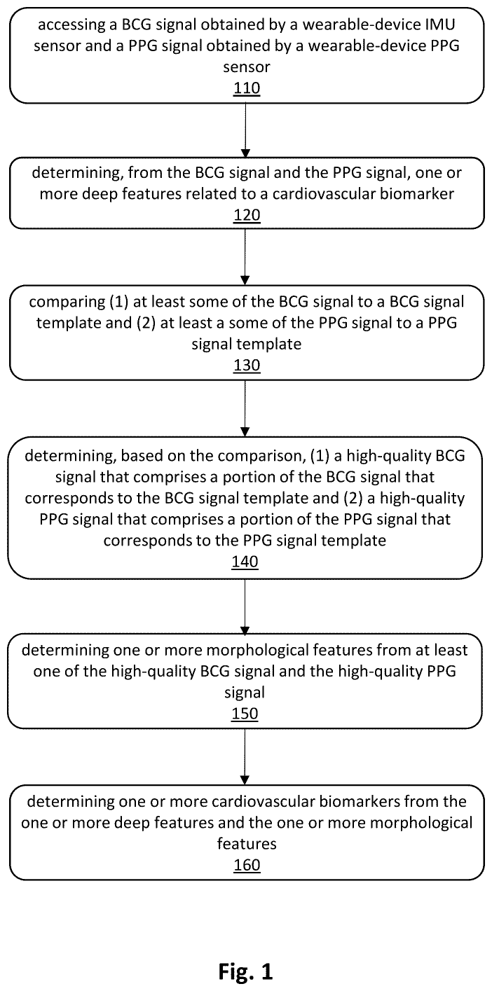

FIG. 1 illustrates an example method for estimating a cardiovascular biomarker from PPG signals and BCG signals obtained from sensors of one or more wearable devices. Step 110 of the example method of FIG. 1 includes accessing a BCG signal obtained by a wearable-device IMU sensor and a PPG signal obtained by a wearable-device PPG sensor. In particular embodiments, these sensors may be in the same wearable device (e.g., may both be in an earbud, a ring, a smartwatch, etc.), while in other embodiments each sensor may be in a different wearable device (e.g., one sensor may be in a smartwatch while another is in an earbud, etc.). In particular embodiments, accessing the sensor signals I step 110 includes obtaining the respective BCG and PPG signals by their respective sensors. In other embodiments, step 110 can include accessing the sensor signals from a device or a device memory (e.g., accessing those signals from the wearable device(s) that hosts the sensors, or accessing those signals from a memory of a client device or a server device, etc.).

Step 120 of the example of FIG. 1 includes determining, from the BCG signal and the PPG signal, one or more deep features related to a cardiovascular biomarker. In particular embodiments the biomarker may be cardiac output, and while several examples of this disclosure relate to detecting cardiac output, other biomarkers may be detected.

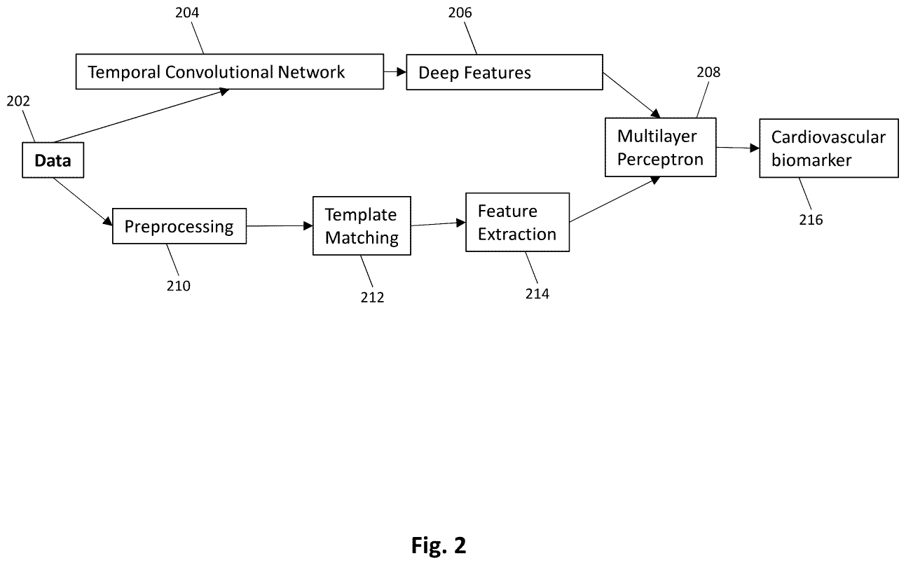

In particular embodiments, step 120 includes extracting deep features from the PPG and/or BCG signals using a trained temporal convolution network (TCN), which is particularly suited to time-series modeling. FIG. 2 illustrates an example implementation of FIG. 1 that uses a trained temporal convolution network. In the example of FIG. 2, data 202 (BCG data, PPG data, or both) is accessed and then passed to temporal convolutional network 204, which extracts deep features 206 for subsequent processing by multilayer perceptron 208. In contrast to traditional convolutional networks, TCN employs causal convolution to ensure temporal consistency. This can be useful in health applications because respecting causal relationships ensures the paramount safety of users (e.g., using future data for prediction might lead to harmful intervention). TCN also employs dilated kernels to effectively capture longer dependencies while maintaining computational efficiency. Training the TCN in the architecture of FIG. 2 in conjunction with the multi-layer perceptron is described more fully below. And as described below, the deep features extracted from the TCN may be fused with interpretable morphological features, e.g., using custom guidance functions described below, to estimate the cardiovascular biomarker. In particular embodiments, extracted deep features d may be represented as d∈ [symbol] where n=128.

Step 130 of the example method of FIG. 1 includes comparing (1) at least some of the BCG signal to a BCG signal template and (2) at least a some of the PPG signal to a PPG signal template. Step 140 then includes determining, based on the comparison, (1) a high-quality BCG signal that comprises a portion of the BCG signal that corresponds to the BCG signal template and (2) a high-quality PPG signal that comprises a portion of the PPG signal that corresponds to the PPG signal template. FIG. 2 illustrates an example implementation of this process. First, data 202 may be preprocessed 210, for example by using filtering (e.g., band-pass filtering) to remove motion artifacts in the data. Particular embodiments employ a Butterworth band pass filter with low and high pass frequencies of 0.7 and 3.5 Hz respectively to remove potential head motion and respiration-caused motion from the wearable-device sensor data.

Step 212 of FIG. 2 includes template matching to select good quality BCG and/or PPG waves. For instance, step 212 may include segmenting wave cycles from the processed sensor data, based on peak detection, and then calculating an average template over all wave cycles in the segment. For instance, sensor data may be divided into segments, and peak detection on the sensor signal in a given segment may be used to identify BCG or PPG wave cycles, respectively. These wave cycles may then be averaged to create a template for that segment. Step 140 may then be performed by selecting the wave cycles in the segment that have high correlations (e.g., greater than 0.9 correction, although other quality threshold levels may be used) with the template. In particular embodiments, a template may be created and stored for future use, i.e., the template for a segment need not be derived from that segment's data in all embodiments, although some embodiments may employ that technique.

Step 150 of the example method of FIG. 1 includes determining one or more morphological features from at least one of the high-quality BCG signal and the high-quality PPG signal. For example, step 214 of FIG. 2 includes extracting morphological features based on the PPG fiducial points or BCG peaks. In particular embodiments, morphological features may include morphological time- and amplitude-wise features. Morphological features may include, but are not limited to, pulse amplitude, pulse time, systolic time, and peak time for PPG signals and pulse amplitude, I-K time difference, I-J time difference, and J-K time difference for BCG signals. Such features are explainable, unlike deep features discussed above. In particular embodiments, extracted morphological features s may be represented as s∈ [symbol] where m=8.

Step 160 of the example method of FIG. 1 includes determining one or more cardiovascular biomarkers from the one or more deep features and the one or more morphological features. For example, FIG. 2 illustrates that deep features 206 and morphological features 214 are passed to multilayer perceptron neural network (MLP) 208, which fuses these features to predict one or more cardiovascular biomarkers 216, e.g., cardiac output. In particular embodiments, MLP 208 may use a custom guidance function to estimate the cardiovascular biomarkers from this input.

For instance, a multilayer perceptron's loss function is usually computed as mean squared error or mean absolute error between prediction and target classes. However, while deep features are obtained through powerful representation learning, deep features lack interpretability and can be subject to noise. On the other hand, surrogate features (i.e., morphological features) are normally derived based on domain knowledge and are validated in clinical environments due to interpretability. Thus, these features can be treated as complementary and fusing can benefit biomarker estimation. Therefore, the following fusion method is used to inherit the prior knowledge from morphological features and guide deep feature in the right direction for biomarker estimation, improving both accuracy and interpretability for biomarker estimation.

Particular embodiments of MLP 208 may fuse deep features and morphological features by using a fusion method that guides deep features to the direction that is related to biomarker (e.g., cardiac output) variation by considering the changing direction of surrogate biomarkers (a potential direction to optimize). For instance, using cardiac output (CO) as the example biomarker, the cardiac output Ĉ may be modeled as a multivariate function: Ĉ=f_{θ}(x), where f_{θ}(·) is the approximated function (parameterized by θ) and x=(d, s) ∈ [symbol] is the feature vector that contributes to estimate CO. By first-order Taylor approximation, the change Δ^{guide} can be estimated as follows:

$${Δ}^{guide} \approx {\sum }_{k = 1}^{m} \frac{\partial {f}_{θ}}{\partial ( {s}^{k} )} \cdot ( {s}_{i}^{k} - {s}_{i - 1}^{k} )$$

where

$${s}_{i}^{k}$$

is the K^{th} Surrogate biomarker (e.g., PPG wave amplitude) extracted the i^{th} signal instance, and

$$\frac{\partial {f}_{θ}}{\partial ( {s}^{k} )}$$

is calculated through the automatic differentiation of a deep learning framework. Next, the direction, Δ^{guide}, from prior knowledge can be integrated as an optimization objective to guide the deep feature learning process as follows:

$${ℒ}^{guide} = \frac{1}{B - 1} {\sum }_{i = 2}^{B} {\vert {\hat{C}}_{i} - ( {\hat{C}}_{i - 1} + {Δ}^{guide} \vert}^{2}$$

where B (set to 256, for example) is the number of input signal pairs. Along with conventional supervision,

$${ℒ}^{m s e} = \frac{1}{B} {\sum }_{i = 1}^{B} {\vert {C}_{i} - {\hat{C}}_{i} \vert}^{2}$$

where Cᵢ is the ground truth for i^{th} signal instance, the objective of MLP 208 is defined below:

$$ℒ = {ℒ}^{m s e} + {βℒ}^{g u i d e}$$

where β is the weight parameter for the guidance loss and may take a value of, e.g., 1.0. This custom objective function tries to learn more relevant deep features and explainable features by guiding the optimization direction based on prior knowledge (i.e., based on surrogate features), unlike the standard objective functions that are based only on MSE between predicted and target values.

MLP 208 is trained using its objective function, such as the custom objective function described above. In particular embodiments, once the MLP is trained on training data, then TCN 204 may subsequently be trained to accurately identify deep features.

The steps of the method of FIG. 1 may be performed by a single device (e.g., a wearable device, a client device, or a server device) or may be performed by a combination of devices. For example, a client device such as a smartphone or personal computer may access sensor data and perform the steps of FIG. 1 locally. In other embodiments, certain steps of FIG. 1 may be performed by a server device, either exclusively in connection with a wearable device, a client device, or both. For instance, a client device may perform steps 110 and 130-150 in particular embodiments, while steps 120 and 160 may be performed by a server device hosting the respective AI models, which may then communicate the biomarker to a client device. In other embodiments, a client device (or less commonly, a wearable device) may host AI models for performing either or both of these steps.

The example method of FIG. 1 uses both BCG data and PPG data to estimate a biomarker. While this approach improves accuracy, it may not always be possible to collect PPG and BCG data at the same time. For example, sampling both PPG and IMU sensors at the same time is more power intensive than using only one sensor. In addition, deep models might not always be the first choice for deploying on client-side devices or on wearable devices. Thus, particular embodiments may use only PPG data or only BCG data to estimate a biomarker, such as cardiac output. These techniques may be used as an alternative to, or in addition to, the technique of FIG. 1. For example, particular embodiments may at times use the technique of FIG. 1 and at other times use PPG-only or BCG-only techniques, e.g., based on available battery life or power demands of a wearable device, etc.

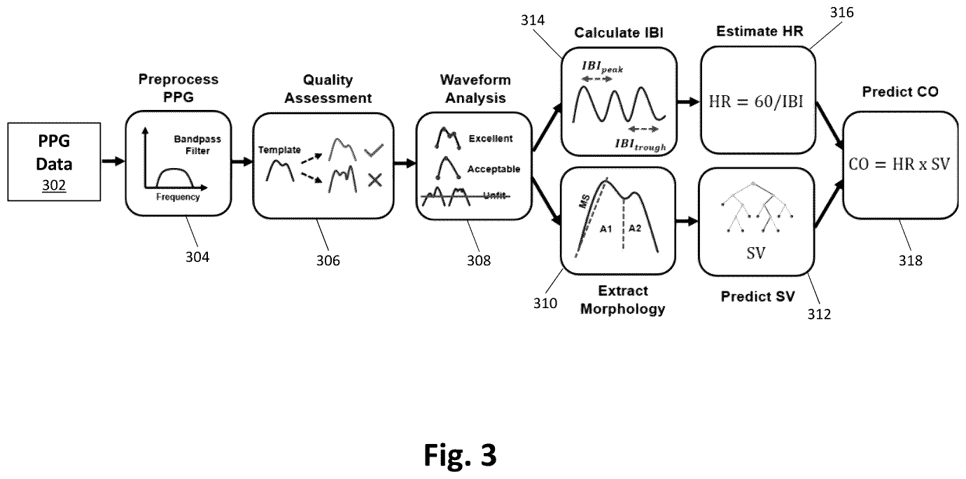

FIG. 3 illustrates an example process for estimating cardiac output from PPG data 302 only. After accessing PPG data 302, the process of FIG. 3 processes the PPG data in step 304, for example by applying a bandpass filter (e.g., 0.7 to 8 Hz, or 0.7 to 2.1 Hz or to 3.5 Hz, etc.) that preserves the PPG morphology. Then, signal quality assessment 306 is performed. For example, PPG data may be segmented, and for each PPG segment, PPG signals can be extracted to derive individual PPG beats. These PPG beats may then be used to derive an average of the PPG waves within their given segment. Individual PPG wave in a segment above a quality threshold, e.g., above 0.9 correlation with the average wave in that segment, are retained for further analysis, while individual PPG waves below the quality threshold in a segment are discarded.

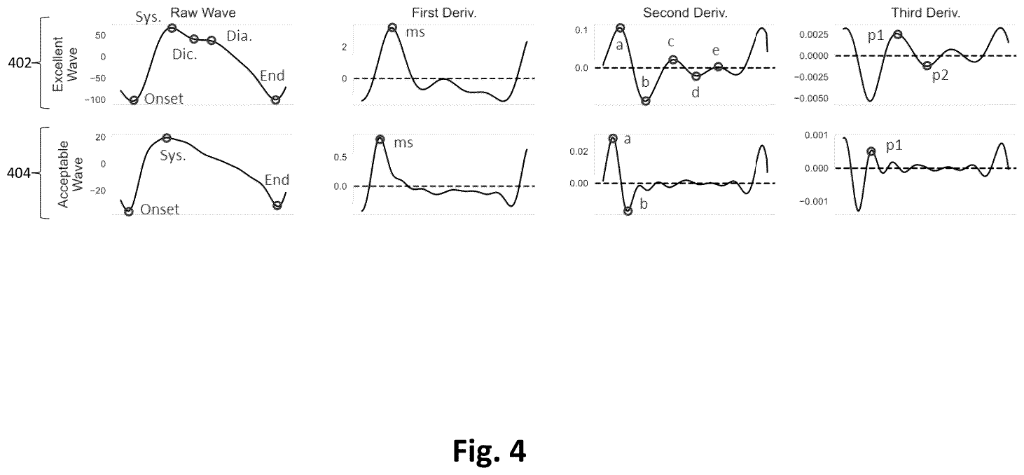

In the example of FIG. 3, passing quality assessment 306 does not mean that the wave will necessary be used for CO prediction. Instead, FIG. 3 employs waveform analysis 308 to determine how each wave that passes quality assessment 306 will be used for downstream CO estimation. For instance, fiducial points in the PPG waveforms may first be detected. Fiducial points in the original wave include the onset, systolic peak (sys), dicrotic notch (dic), diastolic peak (dia), and end, for example as illustrated in FIG. 4. Fiducial points in the first derivative of the wave signal only include the maximum upslope of the wave (ms). Fiducial points in the second derivative of the original wave signal include the early systolic positive wave (a), the early negative systolic wave (b), late re-increasing systolic wave (c), late re-decreasing systolic wave (d), and the dicrotic notch wave (e). Fiducial points in the third derivative include the early systolic component (p₁) and the late systolic component (p₂), as illustrated in FIG. 4. Particular embodiments detect fiducial points for each wave by calculating the zero-crossing points of a given wave's derivative.

In particular embodiments, each wave form that passes quality assessment 306 may be classified into, e.g., one of three categories, such as highest-quality category “Excellent”, a second-highest category “Acceptable”, and low-quality category “Unfit”. Other embodiments may use more or fewer categories, and may use different labels for these categories.

In the example of FIG. 4, “excellent” waves 402 contain both systolic and diastolic morphologies, enabling the detection of all original wave fiducial points (onset, sys, dic, dia, end). “Acceptable” waves 404 included only systolic morphology (onset, sys, end), making the dicrotic notch (dic) and diastolic peak (dia) undetectable. “Unfit” waves lacked discernible morphology, preventing reliable fiducial point detection. For instance, “unfit” wave may have unrealistic morphology such as pronounced diastolic morphology (y (dia)>y (sys)) or more than two visible peaks making it difficult to discern the systolic and diastolic morphology. In the example of FIG. 3, unfit waves are excluded from further analysis, even though such waves have passed quality assessment 306.

After waveform analysis and classification 308, the example of FIG. 3 extracts morphological features from the waves that pass this analysis. Particular embodiments extract features from a waveform in step 310 based on how that waveform is classified in step 308. Table I illustrates an example list of features for both acceptable and excellent quality PPG signal waves. In other words, excellent and acceptable features may be extracted from an excellent wave, while only acceptable features are extracted from an acceptable wave. Particular embodiments determine features per each minute-long PPG segment by calculating the mean feature value cross all waves for that given segment, although other segment lengths may be used.

TABLE I
MORPHOLOGICAL FEATURES WHICH CAN BE EXTRACTED FROM PPG
BASED ON WAVE QUALITY

| Source | Quality | Feature | Definition |
|---|---|---|---|
| Amplitude | Excellent | AI | $\frac{y ( {p}_{2} ) - y ( {p}_{1} )}{y ( sys ) - y ( onset )}$ |
| | | RI | $\frac{y ( dia ) - y ( 0 )}{y ( sys ) - y ( 0 )}$ |
| | | RIₚ₁ | $\frac{y ( dia ) - y ( 0 )}{y ( sys ) - y ( p 1 )}$ |
| | | RIₚ₂ | $\frac{y ( dia ) - y ( 0 )}{y ( sys ) - y ( p 2 )}$ |
| | | ratio_{p1,p2} | $\frac{y ( p 2 ) - y ( 0 )}{y ( p 1 ) - y ( 0 )}$ |
| | | d − a | y″ (d) − y″ (a) |
| | | e − a | y″ (d) − y″ (a) |
| | | AGI | $\frac{{y}^{″} ( b ) - {y}^{″} ( c ) - {y}^{″} ( d ) - {y}^{″} ( e )}{{y}^{″} ( a )}$ |
| | Acceptable | Pulse Amp. | y(sys) − y(onset) |
| | & Excellent | MS Amp. | y′ (ms) − y(onset) |
| | | b − a | y″ (b) − y″ (a) |
| | | c − a | y″ (c) − y″ (a) |
| Area & | Excellent | A1 | A(t(onset), t(dic)) |
| Slope | | A2 | A(t(dic), t(end)) |
| | | IPA | A2/A1 |
| | | IPAD | A2/A1 + d − a |
| | Acceptable & Excellent | MS | $\frac{{y}^{′} ( ms )}{y ( sys ) - y ( onset )}$ |

In Table I, A represents area, y represents amplitude, the ′ symbol represents derivative, and t represents timing.

To predict CO, the example of FIG. 3 first predicts stroke volume (SV) in step 312. For instance SV may be predicted using random forest models trained on extracted morphological features. HR (heart rate) estimate 316 is performed based on calculating the IBI (inter-beat interval) in step 314 from the waveforms in step 308. Then, CO prediction is performed in step 318 as CO=SV*HR. Particular embodiments may estimate CO from each non-overlapping minute window of PPG data, although other window sizes may be used.

Similar to that described above with respect to FIG. 1, the steps of FIG. 3 may be performed by a single device (e.g., a wearable device, client device, or server) device or by a combination of devices, in which each device in the combination performs one or more steps of the process of FIG. 3.

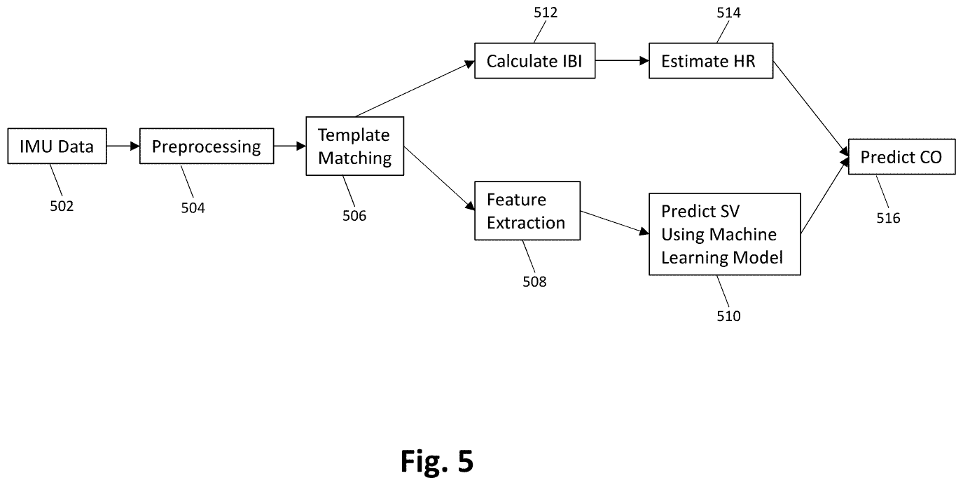

FIG. 5 illustrates a process for estimating CO based only on BCG data, making this process more lightweight than the dual-modality approach of FIG. 1. In the example of FIG. 5, IMU data 502 is accessed and the preprocessed in step 504, for example by applying a bandpass filter (e.g., 0.5 to 20 Hz) that preserves the BCG morphology. Template matching 506 includes segmenting the BCG data and then, for each BCG segment (BCG j-peak to j-peak), BCG signals are extracted to derive individual BCG beats. Then these BCG beats can be used to derive a template, e.g., an average of the BCG waves within their given segments. Individual waves in a segment that exceed a quality threshold, e.g., a 0.9 or higher correlation with the average wave template, are then kept for further analysis, while waves that do not meet this threshold are discarded.

In the example of FIG. 5, feature extraction 508 includes extracting features from a window of BCG data, e.g., a 1-minute window, although other window lengths may be used. Features that can be extracted from BCG data include, but are not limited to: HR, IBI mean, IBI median, RMSSD, SDNN, pNN50, I-J time difference, I-K time difference, J-K time difference, I-J amplitude difference, I-K amplitude difference, J-K amplitude difference, RSA, If/hf ratio, ensemble peak amplitude, average peak amplitude, ensemble RMS energy, and average RMS energy. Then, step 510 includes predicting stroke volume (SV) using, e.g., supervised machine-learning models. The ML model can be trained, for example, on n-1 subjects and tested on the nth subject, and such models may include Bagging (e.g., random forest) or Boosting-based (e.g., gradient boosting, extreme gradient boosting) techniques.

In the example of FIG. 5, heart rate is estimated in step 514 using IBI calculated in step 512, where the IBI can be determined from BCG J-peaks and HR per minute can then be estimated by dividing 60 with the estimated IBI. Finally, CO is predicted in step 516 as the product of HR and SV.

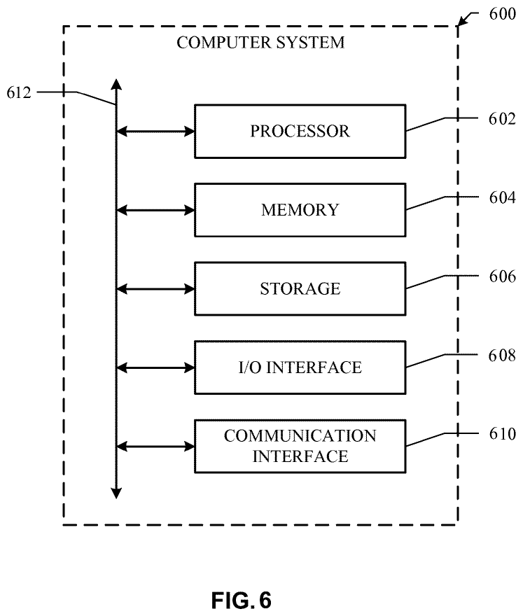

FIG. 6 illustrates an example computer system 600. In particular embodiments, one or more computer systems 600 perform one or more steps of one or more methods described or illustrated herein. In particular embodiments, one or more computer systems 600 provide functionality described or illustrated herein. In particular embodiments, software running on one or more computer systems 600 performs one or more steps of one or more methods described or illustrated herein or provides functionality described or illustrated herein. Particular embodiments include one or more portions of one or more computer systems 600. Herein, reference to a computer system may encompass a computing device, and vice versa, where appropriate. Moreover, reference to a computer system may encompass one or more computer systems, where appropriate.

This disclosure contemplates any suitable number of computer systems 600. This disclosure contemplates computer system 600 taking any suitable physical form. As example and not by way of limitation, computer system 600 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement.

In particular embodiments, processor 602 includes hardware for executing instructions, such as those making up a computer program. As an example and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In particular embodiments, processor 602 may include one or more internal caches for data, instructions, or addresses. This disclosure contemplates processor 602 including any suitable number of any suitable internal caches, where appropriate. As an example and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602. Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In particular embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In particular embodiments, memory 604 includes main memory for storing instructions for processor 602 to execute or data for processor 602 to operate on. As an example and not by way of limitation, computer system 600 may load instructions from storage 606 or another source (such as, for example, another computer system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In particular embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In particular embodiments, one or more memory management units (MMUs) reside between processor 602 and memory 604 and facilitate accesses to memory 604 requested by processor 602. In particular embodiments, memory 604 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory, this disclosure contemplates any suitable memory.

In particular embodiments, storage 606 includes mass storage for data or instructions. As an example and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computer system 600, where appropriate. In particular embodiments, storage 606 is non-volatile, solid-state memory. In particular embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In particular embodiments, I/O interface 608 includes hardware, software, or both, providing one or more interfaces for communication between computer system 600 and one or more I/O devices. Computer system 600 may include one or more of these I/O devices, where appropriate. One or more of these I/O devices may enable communication between a person and computer system 600. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In particular embodiments, communication interface 610 includes hardware, software, or both providing one or more interfaces for communication (such as, for example, packet-based communication) between computer system 600 and one or more other computer systems 600 or one or more networks. As an example and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it. As an example and not by way of limitation, computer system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In particular embodiments, bus 612 includes hardware, software, or both coupling components of computer system 600 to each other. As an example and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing (1) a BCG signal created by a wearable-device IMU sensor and (2) a PPG signal created by a wearable-device PPG sensor;
determining, from the BCG signal and the PPG signal, one or more deep features related to a cardiovascular biomarker;
comparing (1) at least some of the BCG signal to a BCG signal template and (2) at least a some of the PPG signal to a PPG signal template;
determining, based on the comparison, (1) a high-quality BCG signal that comprises a portion of the BCG signal that corresponds to the BCG signal template and (2) a high-quality PPG signal that comprises a portion of the PPG signal that corresponds to the PPG signal template;
determining one or more morphological features from at least one of the high-quality BCG signal and the high-quality PPG signal; and
determining one or more cardiovascular biomarkers from the one or more deep features and the one or more morphological features.

2. The method of claim 1, wherein the one or more deep features are determined by a trained temporal convolutional network.

3. The method of claim 1, further comprising:
segmenting the BCG signal and the PPG signal into a plurality of wave cycles; and
determining (1) the BCG signal template by determining an average wave cycle over the plurality of BCG wave cycles and (2) the PPG signal template by determining an average wave cycle over the plurality of PPG wave cycles.

4. The method of claim 3, wherein (1) the high-quality BCG signal comprises each BCG wave cycle that has a correlation above a threshold with the BCG signal template and (2) the high-quality PPG signal comprises each PPG wave cycle that has a correlation above the threshold with the PPG signal template.

5. The method of claim 1, wherein determining the one or more cardiovascular biomarkers comprises determining the one or more cardiovascular biomarkers by a multi-layer perceptron trained on an objective function comprising a deep-learning loss function that includes a guidance term based on a changing direction of at least some of the one or more morphological features.

6. The method of claim 1, wherein the one or more cardiovascular biomarkers comprise a cardiac output.

7. The method of claim 1, further comprising determining the one or more cardiovascular biomarkers from only a PPG signal, comprising:
accessing a subsequent PPG signal obtained by the wearable-device PPG sensor;
determining a high-quality PPG signal from the subsequent PPG signal;
classifying each PPG waveform in the high-quality PPG signal;
extracting morphological features from each PPG waveform based on that waveform's classification; and
determining the one or more biomarkers from the extracted morphological features.

8. The method of claim 7, wherein extracting morphological features from each PPG waveform based on that waveform's classification comprises:
extracting both systolic and diastolic features from each PPG waveform receiving a highest-quality classification;
extracting only systolic features from each PPG waveform receiving a second-highest quality classification; and
extracting no features from each PPG waveform receiving a lowest-quality classification.

9. The method of claim 1, further comprising determining the one or more cardiovascular biomarkers from only a BCG signal, comprising:
accessing a subsequent BCG signal obtained by the wearable-device BCG sensor;
segmenting the subsequent BCG signal into a plurality of wave cycles;
determining, for each segment, a BCG signal template by determining an average wave cycle over the plurality of BCG wave cycles;
determining a high-quality BCG signal comprising each BCG wave cycle that has a correlation with the BCG signal template that is greater than a threshold;
extracting BCG features from a window of high-quality BCG signals; and
determining the one or more biomarkers from the extracted BCG features.

10. A system comprising one or more non-transitory computer readable storage media storing instructions, and one or more processors coupled to the one or more non-transitory computer readable storage media and operable to execute the instructions to:
access a (1) a BCG signal created by a wearable-device IMU sensor and (2) a PPG signal created by a wearable-device PPG sensor;
determine, from the BCG signal and the PPG signal, one or more deep features related to a cardiovascular biomarker;
compare (1) at least some of the BCG signal to a BCG signal template and (2) at least a some of the PPG signal to a PPG signal template;
determine, based on the comparison, (1) a high-quality BCG signal that comprises a portion of the BCG signal that corresponds to the BCG signal template and (2) a high-quality PPG signal that comprises a portion of the PPG signal that corresponds to the PPG signal template;
determine one or more morphological features from at least one of the high-quality BCG signal and the high-quality PPG signal; and
determine one or more cardiovascular biomarkers from the one or more deep features and the one or more morphological features.

11. The system of claim 10, wherein the one or more deep features are determined by a trained temporal convolutional network.

12. The system of claim 10, further comprising one or more processors that are operable to execute the instructions to:
segment the BCG signal and the PPG signal into a plurality of wave cycles; and
determine (1) the BCG signal template by determining an average wave cycle over the plurality of BCG wave cycles and (2) the PPG signal template by determining an average wave cycle over the plurality of PPG wave cycles.

13. The system of claim 12, wherein (1) the high-quality BCG signal comprises each BCG wave cycle that has a correlation above a threshold with the BCG signal template and (2) the high-quality PPG signal comprises each PPG wave cycle that has a correlation above the threshold with the PPG signal template.

14. The system of claim 10, wherein determining the one or more cardiovascular biomarkers comprises determining the one or more cardiovascular biomarkers by a multi-layer perceptron trained on an objective function comprising a deep-learning loss function that includes a guidance term based on a changing direction of at least some of the one or more morphological features.

15. The system of claim 10, wherein the one or more cardiovascular biomarkers comprise a cardiac output.

16. The system of claim 10, further comprising one or more processors that are operable to execute the instructions to determine the one or more cardiovascular biomarkers from only a PPG signal by:
accessing a subsequent PPG signal obtained by the wearable-device PPG sensor;
determining a high-quality PPG signal from the subsequent PPG signal;
classifying each PPG waveform in the high-quality PPG signal;
extracting morphological features from each PPG waveform based on that waveform's classification; and
determining the one or more biomarkers from the extracted morphological features.

17. The system of claim 16, wherein determining the high-quality PPG signal comprises:
extracting, from the subsequent PPG signal, a plurality of PPG beats;
determining, from the plurality of PPG beats, an average PPG beat;
comparing each of the plurality of PPG beats to the average PPG beat; and
creating the high-quality PPG signal by including in the high-quality PPG signal only those PPG beats that have a greater-than-threshold correlation with the average PPG beat.

18. The system of claim 16, wherein extracting morphological features from each PPG waveform based on that waveform's classification comprises:
extracting both systolic and diastolic features from each PPG waveform receiving a highest-quality classification;
extracting only systolic features from each PPG waveform receiving a second-highest quality classification; and
extracting no features from each PPG waveform receiving a lowest-quality classification.

19. The system of claim 10, further comprising one or more processors that are operable to execute the instructions to determine the one or more cardiovascular biomarkers from only a BCG signal, comprising:
accessing a subsequent BCG signal obtained by the wearable-device BCG sensor;
segmenting the subsequent BCG signal into a plurality of wave cycles;
determining, for each segment, a BCG signal template by determining an average wave cycle over the plurality of BCG wave cycles;
determining a high-quality BCG signal comprising each BCG wave cycle that has a correlation with the BCG signal template that is greater than a threshold;
extracting BCG features from a window of high-quality BCG signals; and
determining the one or more biomarkers from the extracted BCG features.

20. One or more non-transitory computer-readable storage media comprising instructions that are operable when executed by one or more processors to:
access a (1) a BCG signal created by a wearable-device IMU sensor and (2) a PPG signal created by a wearable-device PPG sensor;
determine, from the BCG signal and the PPG signal, one or more deep features related to a cardiovascular biomarker;
compare (1) at least some of the BCG signal to a BCG signal template and (2) at least a some of the PPG signal to a PPG signal template;
determine, based on the comparison, (1) a high-quality BCG signal that comprises a portion of the BCG signal that corresponds to the BCG signal template and (2) a high-quality PPG signal that comprises a portion of the PPG signal that corresponds to the PPG signal template;
determine one or more morphological features from at least one of the high-quality BCG signal and the high-quality PPG signal; and
determine one or more cardiovascular biomarkers from the one or more deep features and the one or more morphological features.
